(19) United States
(12) Patent Application Publication
(10) Pub. No.: US 2021/0000509 A1
(43) Pub. Date: Jan. 7, 2021

(54) PIN FASTENER WITH REMOVEABLE DRILL BIT FOR BONE FIXATOR

(71) Applicants: Scot HODKIEWICZ, Lake Geneva, WI (US); Jeremy UTKE, Osceola, WI (US)

(72) Inventors: Scot HODKIEWICZ, Lake Geneva, WI (US); Jeremy UTKE, Osceola, WI (US)

(21) Appl. No.: 16/976,641

(22) PCT Filed: Feb. 12, 2019
(86) PCT No.: PCT/US2019/017677

§ 371 Date: Aug. 28, 2020

Related U.S. Application Data
(60) Provisional application No. 62/637,783, filed on Mar. 2, 2018.

(51) Int. Cl. A61B 17/64; A61B 17/16; A61B 17/72; A61B 17/86

(52) U.S. Cl. CPC A61B 17/6475; A61B 17/1615; A61B 17/72; A61B 17/848; A61B 2017/8655; A61B 17/864; A61B 17/86

(57) ABSTRACT

A fixator pin with a removable drill bit configured for simultaneously drilling a hole into bone for a fixator pin and threading or installing the fixator pin into the hole. The drill bit portion is joined to the external fixator pin via a separable connection configured as a cut-off, a frangible connection, brittle snap section, or threaded connection. The frangible connection includes at least one feature formed through an outer surface of the frangible connection. The frangible connection is configured to promote the selective separation of the drill bit from the fixator portion at the feature, which may not be separated in the case of an intramedullary pin or nail.

### BACKGROUND OF THE INVENTION

### Field of the Invention

The present invention relates to external fixators, intramedullary pins, and nails, and an improved method of fixation.

### Relevant Technology

It is well known that fractured bones may be effectively healed by fixing the fractured bone in a secure position to prevent slippage or separation. With a bone so secured, bone tissue will grow and bone cells will multiply in the region of the fracture.

One common technique for securing fractures is an external fixation pin or screw that extends into or through the bone fragments to secure the fragments in a fixed position. Typically, the fixation pin pierces the outer cortex of the fractured bone, crosses the medullary canal and embeds in or passes through the opposite cortex.

Another technique is the use of intramedullary pins (also known an IM pin) or intramedullary nail (also known as an IM nail, or inter-locking nail, or Küntscher nail), that are driven through the bone and across the fracture. Intramedullary pins tend to be round where as nails tend to have a series of holes through the proximal and distal portion of the nail through which a series of pins are driven through the bone and through the nail to further strengthen the construct.

Like plates and screws, external fixators, intramedullary pins, or nails provide strong support for broken bones. External fixators, intramedullary pins, or nails provide the advantage of minimal tissue disruption which allows for rapid healing. Unlike plates or pins, part of the external fixator sits outside the body; pins run from the bone to outside the skin where they connect to a weight bearing rod.

External fixation involves placing either threaded or unthreaded pins percutaneously through the skin and overlying tissues and through a bone. The external fixator pin is driven through or screwed into the bone and may or may not exit the skin on the other side opposite the insertion side. Once the fixator pins are set in the bone and the fracture is aligned as desired, the pins are held in place or fixed by a rod, modular device, tubular device, or the like, that is clamped to the pins external to the limb. Recently, the rod and clamp configuration has been replaced in some instances by a polymer that secures the pins external to the limb. The polymer is typically an epoxy or similar product. The apparatus holds the bones in position until healed at which time the entire apparatus is removed.

Typically, a hole is drilled through the bone and the external fixator pin, intramedullary pin, or nail is then placed through this hole. Failure to do so may result in cracking of the bone at the pin site. This is one cause of early pin failure and loosening may result or the bone fragment may shift resulting in a more complicated fracture. Because of this, the surgeon usually drills an initial hole through the bone and overlying tissues. The surgeon then must pull the drill back out, set the drill down and pick up the external fixator pin and then search for the hole that was drilled in order to place the pin through the bone. This can be time consuming and complicated since the overlying tissues are mobile and often obscure the drill hole. The surgeon may be unable to locate the drill hole to place the pin. This may necessitate the redrilling of the hole further weakening the bone and potentially leading to fracture.

Some fixator pins are self-drilling and self-tapping. These pins have smooth, cylindrical shafts with the points matched into a pointed spade configuration which formed the drill tip, knife edges that scraped away the bone when the shaft was turned, and a self-tapping thread. The self-tapping thread continued up the shaft for a distance sufficient to pass through the bone for which the pin was designed.

Self-drilling, self-tapping screws, pins, and nails have several disadvantages. First, the knife edge of the drill point is not very sharp. Consequently, the drill advances at a relatively slow speed through the bone. This slow speed was generally slower than the speed with which the self-tapping thread would advance if the hole were pre-drilled before attempting to tap the pin into the bone. This speed differential caused the thread portion to strip out the threads just cut in the bone because of the inability to advance as fast as the self-tapping thread would normally advance. A second disadvantage is that the relatively slow speed of drill bore progression achieved by this structure resulted in higher temperatures from friction heating of the bone surrounding the hole.

Another self-tapping, self-drilling orthopedic fixation screw includes a cannula through the shaft of the screw. The cannula is placed over a guide pin mounted in a guide hole in a bone to drill and tap a hole at predetermined location in the bone. In this manner the fixation pin is attached to the bone.

### SUMMARY OF THE INVENTION

It is an object of this invention to provide an external fixator pin, intramedullary pin, or intramedullary nail with a removable or non-removable drill portion. The drill portion is removed when no longer necessary leaving the threaded and engaged portion of the external fixator, intramedullary pin, or nail placed within the bone. The removable drill portion would also be applicable to smooth, non-threaded variants of fixator pins allowing quick and easy removal of the sharpened drill bit portion of the fixator. The external fixator pin is useable in all applications including but not limited to unilateral uniplanar applications, uniplanar bilateral applications, bilateral biplanar applications, unilateral biplanar applications, and the like. Both planar and circular external fixators can be used with the disclosed external fixator pins. Similarly, the intramedullary pin or nail may be placed in any direction with respect to the bone.

As used herein, the term fixator pin is used to include an external fixator pin, an intramedullary pin, an intramedullary nail, Schanz screw, inter-locking nail, Küntscher nail, half pins, centrally threaded full pins, Steinmann pins, smooth transfixation pins, positive or negative profile transfixation pins, and the like.

The disclosed external fixator pin allows better purchase and positive thread engagement into the bone. Better purchase is important since the bone is only solid on the outer portion with soft bone or marrow internally. Thus, threads only have purchase on the outer two sides of the bone, referred to as the outer cortices of the bone. The disclosed external fixator pin substantially eliminates the risk of the external fixator threads stripping out of the bone if the drill bit did not move through the second cortex of bone at the same rate as the threaded portion of the fixator.

Combining the drill bit onto the external fixator pin, intramedullary pin, or nail in a removable configuration allows the surgeon to drill the guide hole and set the pin without removing it from the drilled hole, which simplifies the placement of the fixator pin, intramedullary pin, or nail, saving time and reducing complications. The drill bit is a fluted drill bit, a twist drill bit, a brad point bit, a high speed steel bit, a titanium nitride coated bit, a cobalt high speed steel bit, an auger, a spade, or the like.

As discussed above, another problem encountered with prior art pins is that once a hole is drilled through both cortices of the bone and a pin, screw or nail is inserted, the pin, screw or nail may not enter the hole on the far side of the bone. If the external fixator pin, IM pin or IM nail misses the far cortex hole, it often results in cracking of the bone near the drilled hole or a stripping of the threads in the near hole. This problem is eliminated when the drill is combined with the pin or nail.

According to one aspect of the invention, a snap-off or separation point is incorporated between the external fixator pin, intramedullary pin, or nail portion and the drill bit portion that allows simple removal of the drill bit when no longer necessary.

According to one aspect of the invention, a right-handed thread, or a thread that is the same as the drill direction and the fixator pin thread, is incorporated between the external fixator pin portion and the drill bit portion that allows simple removal of the drill bit when no longer necessary.

According to one aspect of the invention, a left-handed thread, or a thread that is opposite the drill direction and the fixator pin thread, is incorporated between the external fixator pin portion and the drill bit portion that allows simple removal of the drill bit when no longer necessary.

According to one aspect of the invention, the external fixator pin is unthreaded, has a positive profile, or a negative profile.

According to one aspect of the invention, a snap-off drill bit comprising a fluted drill bit of various drill designs is joined to the external fixator pin via a frangible connection comprising at least one feature formed through the outer surface of the shaft at a point either near the threaded or non-threaded portion of the external fixator pin in the case of a pin that does not pass through the patient's tissues on the far side of the limb or a suitable distance away from the threaded or non-threaded portion of the external fixator pin for pins that are placed completely through both sides of the limb.

According to one aspect of the invention, a cut-off drill bit comprising a fluted drill bit of various drill designs and lengths is joined to the external fixator pin near the threaded or non-threaded portion of the external fixator pin and removed by a cut-off tool.

According to one aspect of the invention, a machine threaded drill bit comprising a fluted drill bit of various drill designs and lengths is joined to the external fixator pin near the threaded or non-threaded portion of the external fixator pin via internal or external threads or features and removed by unthreading or unscrewing from the external fixator pin.

According to one aspect of the invention, the drill bit, which is removable via a frangible connection, brittle snap off section, threaded features, cut-off tools or processes, and the like, is joined to a threaded fixator pin, a non-threaded fixator pin, an intramedullary pin, an intramedullary rod, or the like.

According to one aspect of the invention, the external fixator pin or nail includes multiple frangible points on either or both the far side of pin or near side of pin to break off at set distance from threads (a set distance away from the threads nearer the surgeon). This would avoid having to cut and grind pin on near side of fracture and give surgeon options for where to break off the pin. This may be useful with external fixator pin or IM pins that can be broken off at a set distance from the pins.

One advantage of this drill/pin configuration is that the drill bit is only used once and then broken or cut off and discarded. This ensures that the drill bit is always sharp easing the creation of the drill hole. Reusable drill bits, which are commonly used in surgery, become dulled with repeated use and so lead to increased friction and heating of the bone. This results in bone death adjacent to the drill hole. This bone is quickly resorbed by the body leading to premature loosening of the pin, increased infection rates, and poor or delayed healing. The use of one-time use drill bits in this embodiment reduces these problems.

### BRIEF DESCRIPTION OF THE DRAWINGS

In the drawings:

FIG. 1. is a broken bone with an external skeletal fixation system;

FIG. 2. is a broken bone with a partially installed external fixator pin of an external skeletal fixation system;

FIG. 3 is a prior art external fixator pin;

FIG. 4 is an external fixator pin;

FIG. 5 is an external fixator pin;

FIG. 6 is an external fixator pin;

FIG. 7 is an external fixator pin;

FIG. 8 is an external unthreaded fixator pin;

FIGS. 9A-9C are IM pins with removable drill bits;

FIGS. 10A-10D are intramedullary nails with a removable drill bits; and

FIG. 11 is a broken bone with a partially installed external fixator pin of an external fixator with smooth and threaded pins.

### DETAILED DESCRIPTION OF THE PRESENTLY PREFERRED EMBODIMENTS

External skeletal fixation is used for primary or secondary stabilization of open or closed long bone fractures, spinal fractures and luxations, luxations or arthrodesis of certain joints, and to provide support following ligament or tendon reconstruction. External skeletal fixation can also be used to dynamically enhance long bone growth following premature physeal closure, and to facilitate and reestablish limb alignment in juvenile patients and select adult patients. External skeletal fixators use percutaneous transfixation implants that may be stainless steel wires (Kirschner wires), pins, or both, coupled with an external frame that may be linear (connecting bars), circular (transfixation pins connected to rings, which are connected by threaded bars), or a hybrid, which is a combination of linear and circular external skeletal fixation elements, and may be placed into various geometric configurations.

External skeletal fixator frames can be composed of a variety of materials such as stainless steel, carbon fiber, titanium, polymers, or acrylics. The final frame design may function as a supportive exoskeleton for fractures and osteotomies, or as rigid immobilization for transarticular applications. External skeletal fixation is especially effective with highly complex fractures. While widely used in humans, external skeletal fixators are well tolerated by animals including dogs and cats, allowing early return to limb function following fracture fixation, and usually can be removed without the need for administration of general anesthesia to the patient. Finally, external skeletal fixation systems are generally more economical for the surgeon and the client. Following cleaning and sterilization, some components can be reused on multiple patients over time.

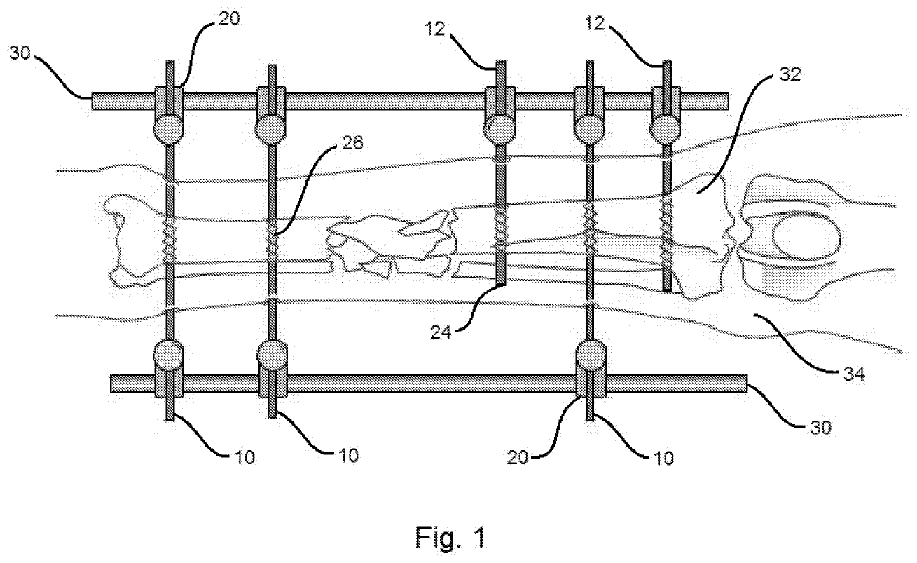

FIG. 1. is a broken bone with an external skeletal fixation system. Collectively, the frame and the transfixed bone are referred to as a construct or montage. As shown in FIG. 1, a broken bone 32 is transfixed using a plurality of external fixator pins. External fixator pins 10 extend completely through both the bone 32 and tissue 34. Both ends of each external fixator pin 10 are attached to a connecting bar 30 via a clamp 20. It should be noted that clamps 20 can be any type of clamp including, but not limited to universal clamps, open ended clamps, transverse pin adjusting clamps, tube to tube clamps, and the like. The external fixator pin 10 is secured to the bone by a threaded portion 26.

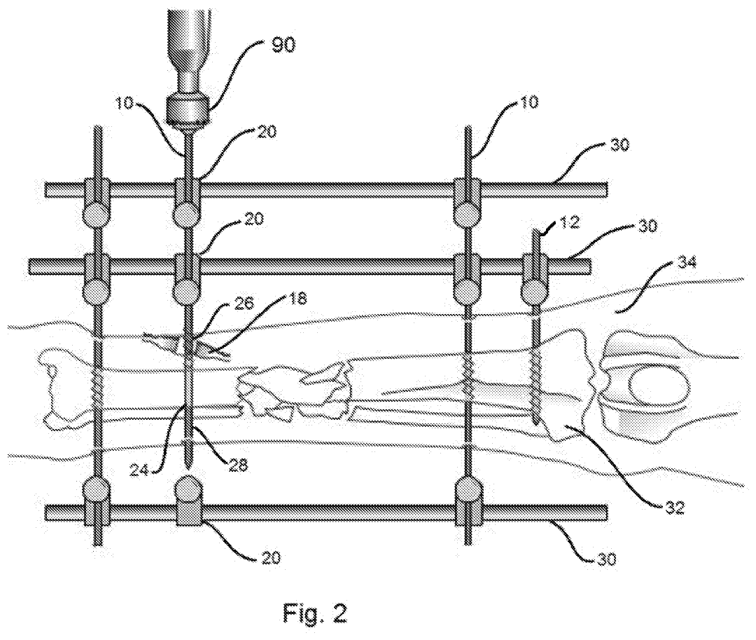

The external fixator pin 10 shown in FIG. 2, which is partially inserted, comprises a removable drill tip 28, preferably including a fluted drill bit, joined to the external fixator pin via a separable connection at a point spaced apart from the threaded or non-threaded portion of the external fixator pin so that the external fixator pin passes through the patient's tissues on the far side of the limb at a suitable distance away from the threaded or non-threaded portion of the external fixator pin so that the pin passes completely through both sides of the limb and the patients tissue.

As shown in FIGS. 1 and 2, external fixator pins 10 extend through both the bone 32 and tissue 34 and external fixator pins 12 do not exit the tissue 34 at both ends. The external fixator pin 12 extends from the tissue 34 only on the side through which it was inserted. The end of the external fixator pin 12 that extends from the tissue 34 is attached to the connecting bar 30 via clamp 20. An end 24 of the external fixator pin 12 extends past the bone 32 and terminates in the tissue 34.

The external fixator pins 10 and 12, shown in FIGS. 1 and 2, each comprise a snap-off drill bit, preferably including a fluted drill bit, joined to the external fixator pin via a separable connection at a point either near the threaded or non-threaded portion of the external fixator pin in the case of a pin that does not pass through the patient's tissues on the far side of the limb. It should be noted that the length of the pin can vary and the connection point or points can be arranged at various locations along the fixator pin. For those fixator pins that do not pass through the tissue on the far side the connection point is proximate to the bone.

FIG. 2. is a broken bone with a partially installed external fixator pin 10 of an external skeletal fixation system. A drill 90 is used to install external fixator pin 10. According to one aspect of the invention, a second connecting bar 30 and clamp 20 is used as a drill guide. The external fixator pin 10 is guided into the limb via an incision 18. As shown, the removable drill tip 28 is still attached to the external fixator pin 10 that is being installed by the drill 90. Once the threaded portion 26 is threaded into the bone 32 via the hole drilled by the removable drill tip 28, the removable drill tip 28 is removed from the external fixator pin 10. Once the removable drill tip 28 is removed from the external fixator pin 10, an end 24 of the external fixator pin 10 remains. The external fixator pin 12 also comprises a removable drill tip 28.

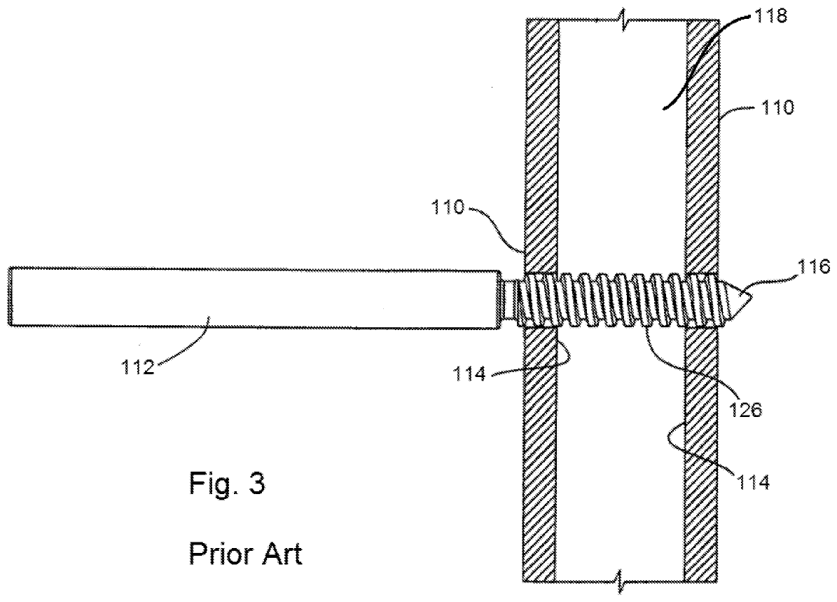

FIG. 3 is an external fixator pin 112 according to the prior art. The external fixator pin 112 penetrates the cortices (outer wall 110 and inner wall 114) and traverses the medullary canal 118 of a bone. A drill tip 116, arranged proximate to the thread 126 is the mechanism used to drill through the bone. Alternatively, the external fixator pin 112 can include a fluted drill portion between the drill tip 116 and the thread 126.

The external fixator pin 112 is installed using the following procedure. After an incision is made in the skin, a sleeve is inserted through the incision and pushed onto the bone. The sleeve is held steady and a trocar is tapped on the bone surface to create an initial impression. The impression prevents slipping of the drill bit during drilling. The trocar is removed and a long drill bit is inserted through the sleeve and both cortices are drilled through. The drill bit is withdrawn and the external fixator pin is introduce through the sleeve threaded into bone so that the thread 126 is securely engaged into the far cortex. The external fixator pin 112 is then attached to a connecting bar 30 via a clamp 20. Alternatively, the external fixator pin 112 is installed without a sleeve or trocar in a two-step process. In the two-step process a hole is drilled with a drill bit. Once the hole through the bone is drilled, the drill bit is removed and the external fixator pin is installed in the hole that was drilled in the bone. This external fixator pin 112 suffers from the limitations discussed above.

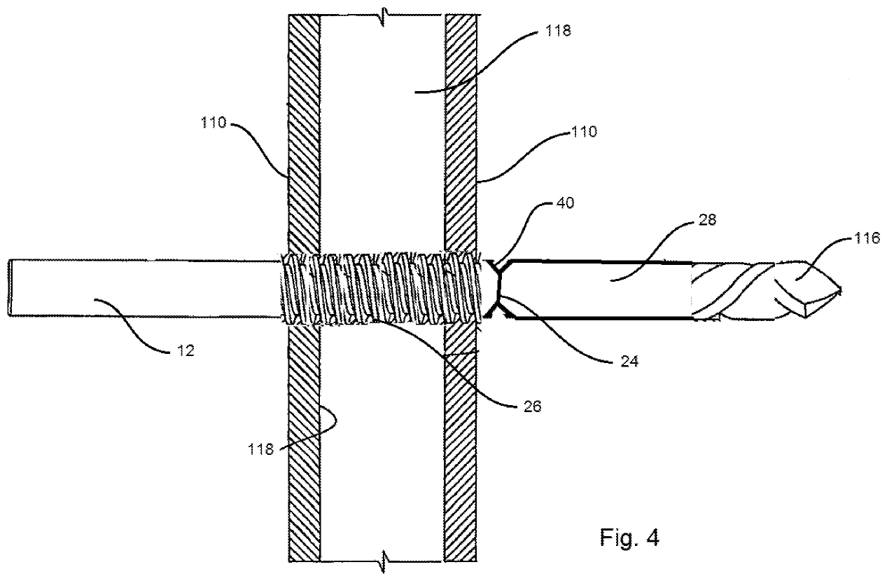

FIG. 4 is an external fixator pin 12 according to one aspect of the invention. As shown, the external fixator pin 12 comprises a shaft configured to be attached to a connecting bar 30 via a clamp 20, as shown in FIG. 2. The external fixator pin 12 further includes a removable drill tip 28, which has a drill tip 116. According to one aspect of the invention, the external fixator pin 12 includes a fluted drill portion proximate to the drill tip 116.

The external fixator pin 12 shown in FIG. 4 is characterized by a frangible connection 40 formed proximate to the threaded portion 26. The frangible connection 40 is formed by a circumferential narrowing of the external fixator pin 12 at a specific location. To break the frangible connection 40, the removable drill tip 28 is bent back and forth until the frangible connection snaps. Alternatively, the frangible connection is configured as a weakened portion, a metallurgically brittle section, a cut, a groove in the external fixator pin 12 substantially perpendicular to a longitudinal axis of the external fixator pin 12, or the like. While the frangible connection 40 is shown as a v-shaped groove, any feature or defect introduced into the external fixator pin 12 at a desired location that will provide the frangible connection 40 that can be used. In one embodiment, the weakened portion is a metallurgically brittle portion. Alternatively, the frangible connection 40 is a cut-off point that is cut using a cut-off tool. Once the removable drill tip 28 is removed from the external fixator pin 12, an end 24 of the external fixator pin 12 remains. An added advantage of the frangible connection 40 is that the removable drill tip 28 realizes maximum drilling performance since it is only used once and then broken off and discarded.

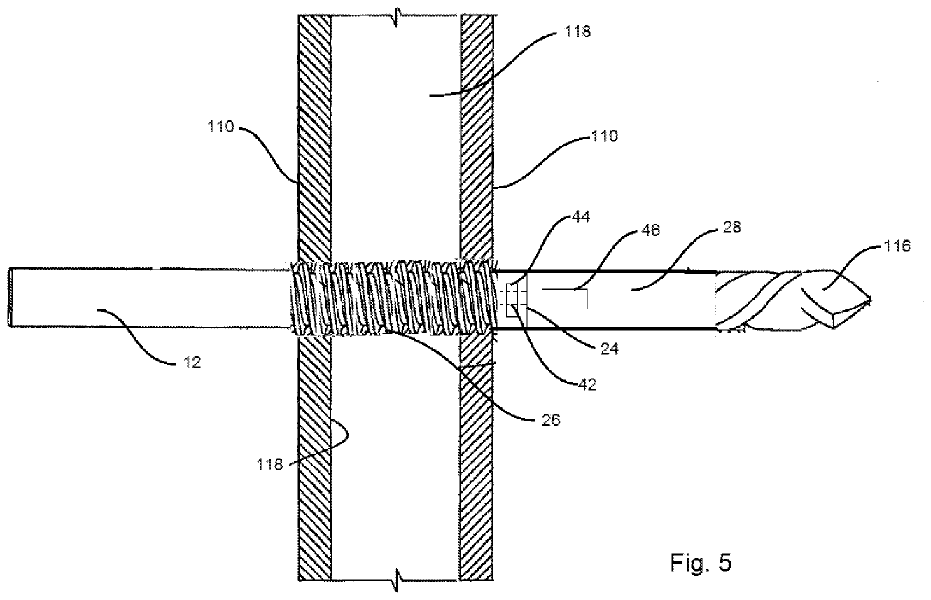

FIG. 5 is an external fixator pin 12 according to one aspect of the invention. Instead of a frangible or cut-off connection between the external fixator pin 12 and the removable drill tip 28, the external fixator pin 12 and the removable drill tip 28 are threadingly connected by a threaded connection 42. As shown, the removable portion has a screw portion that extends therefrom and is threaded into the external fixator pin 12. The external fixator pin 12 can be tapped with a thread or the screw portion of the removable drill tip 28 can be self-tapping. In one embodiment, the blind hole into which the screw portion of the removable drill tip 28 has a radially extending drain hole so that nothing accumulates in the blind hole. While the screw portion is shown as being part of the removable drill tip 28, the screw portion can also be arranged on the external fixator pin 12 and screwed into the removable drill tip 28. The screw thread of the threaded connection 42 can be the same as the threaded portion 26 or opposite the threaded portion 26. Preferably, the screw thread of the threaded connection 42 is same as the threaded portion 26. In other words, if the threaded portion 26 is a right handed thread, the screw thread of the threaded connection 42 is also right handed thread to keep the connection tight during a right handed drilling operation. In one embodiment, the screw thread of the threaded connection 42 is opposite the threaded portion 26. In other words, if the threaded portion 26 is a right handed thread, the threaded connection 42 of the screw portion is a left handed thread. After insertion, the removable portion is unscrewed leaving the external fixator pin.

According to one aspect of the invention shown in FIG. 5, there are flats 44, 46 arranged on the external fixator pin 12 and the removable drill tip 28, respectively. The flats 44, 46 are configured so that they can be grabbed with a wrench, pliers, or the like. It should be noted that the flat 44 on the external fixator pin 12 can be arranged at the distal end opposite the end 24.

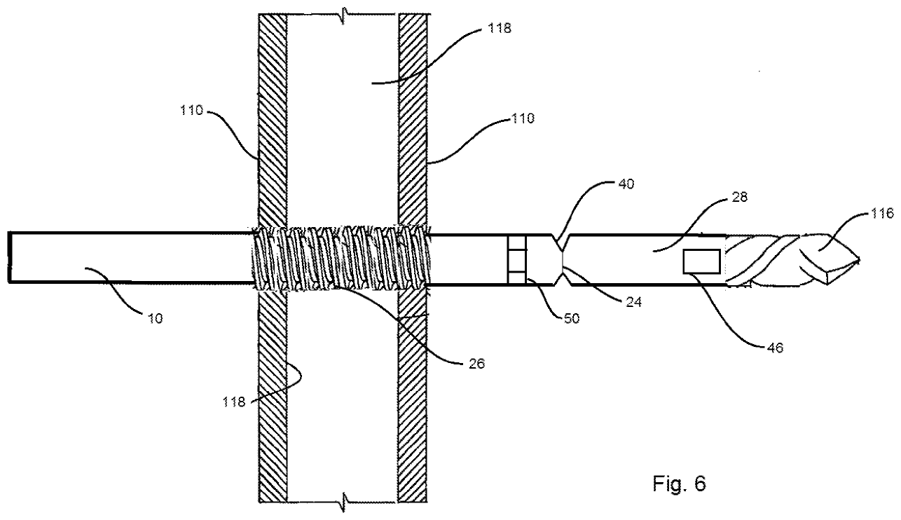

FIG. 6 an external fixator pin 10 according to one aspect of the invention. The external fixator pin 10 includes a frangible connection 40. The external fixator pin 10 extends longitudinally from threaded portion 26 on both ends to pass through the patients tissue so that both ends of the external fixator pin 10 can be secured to respective connecting bars 30. The external fixator pin 10 includes flats 46, 50. The flats can be incorporated into any embodiment of the invention. The flats 50 are configured as a hex nut or the like. The flats can also be arranged at distal ends of the external fixator pin 10. An added advantage of the frangible connection 40 is that the removable drill tip 28 is only used once and then broken off and discarded.

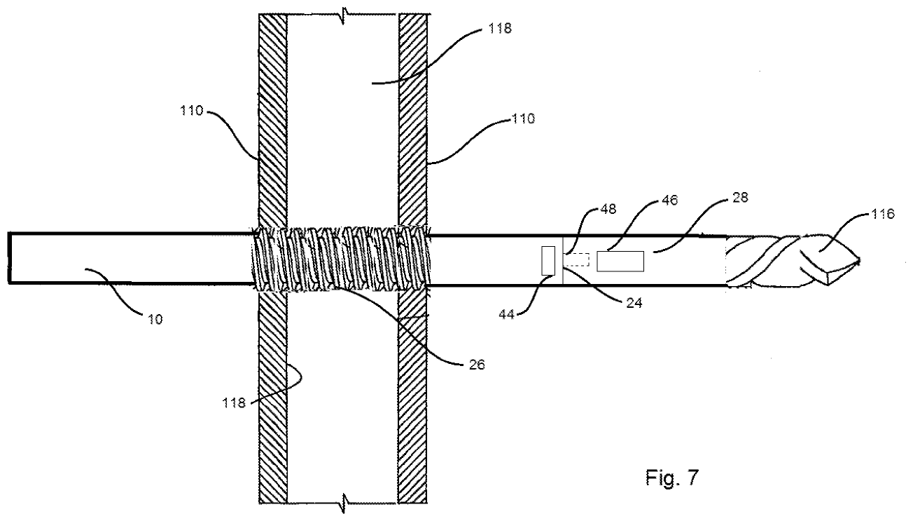

FIG. 7 an external fixator pin 10 according to one aspect of the invention. The external fixator pin 10 and the removable drill tip 28 are threadingly connected by a threaded connection 48. The external fixator pin 10 extends longitudinally from threaded portion 26 on both ends to pass through the patients tissue so that both ends of the external fixator pin 10 can be secured to respective connecting bars 30. The threaded connection shown in FIG. 7 has the screw portion arranged on the external fixator pin 10 and is configured to be screwed into the removable drill tip 28. An added advantage of the threaded connection is that the removable drill tip 28 can be sharpened, sterilized, and reused and the external fixator pin 12 can also be sterilized and reused.

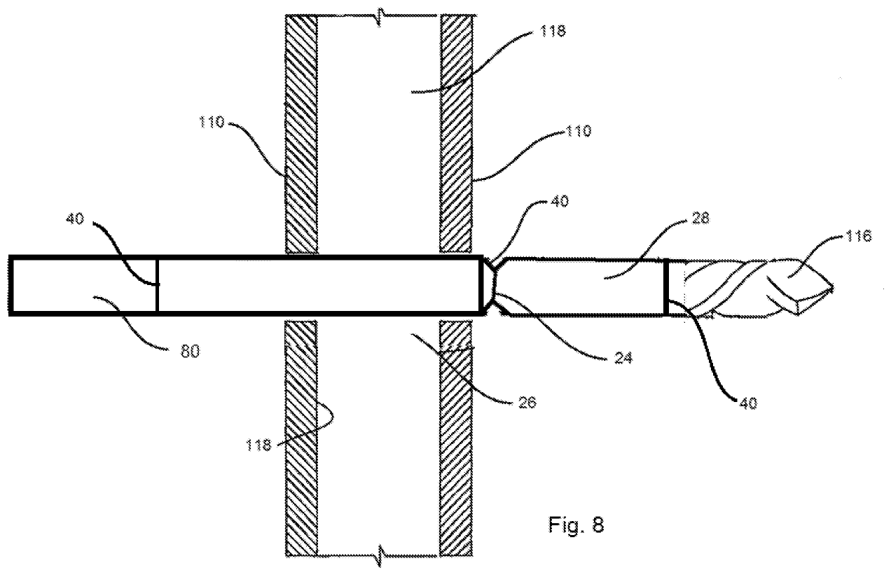

FIG. 8 is an external fixator pin 80 according to one aspect of the invention. As shown, the external fixator pin 80 comprises a shaft configured to be attached to a connecting bar 30, which is shown in FIG. 2. The external fixator pin 80 further includes a removable drill tip 28, which has a drill tip 116. According to one aspect of the invention, the external fixator pin 80 in this figure includes a fluted drill portion proximate to the drill tip 116.

The external fixator pin 80 shown in FIG. 8 is characterized by a frangible connection 40 formed proximate to the threaded portion 26. As shown, a plurality of frangible connections 40 can be incorporated into the external fixator pin at various locations. While the frangible connection 40 is shown as a v-shaped groove, any feature or defect introduced into the external fixator pin 80 at a desired location that will provide the frangible connection 40 can be used. Alternatively, the frangible connection 40 is a cut-off point that is cut using a cut-off tool. Once the removable drill tip 28 is removed from the external fixator pin 80, an end 24 of the external fixator pin 12 remains. An added advantage of the frangible connection 40 is that the removable drill tip 28 realizes maximum drilling performance since it is only used once and then broken off and discarded.

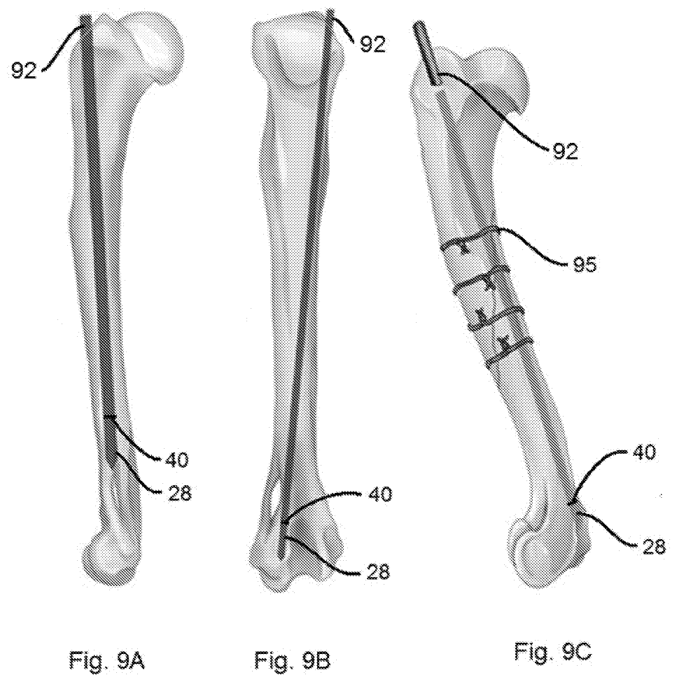

FIG. 9A-9C are intramedullary pins with removable drill bits. The intramedullary pins can be round, triangular, square, V-shaped in cross-section, a clover-leaf shape, or the like. To align fractured bones and provide optimal healing support, a small rod-like nail device, intramedullary pin 92 is inserted into the hollow center of the bone. The intramedullary pin 92 forms a self-contained internal splint to stabilize the fracture. As shown, intramedullary pins 92 pass through a bone longitudinally. Specifically, the intramedullary pins 92 pass through the medullary canal of the bone. Intramedullary pins 92 include a frangible connection 40 and removable drill tip 28. In use, the intramedullary pins 92 are drilled into and through a bone, the removable drill tip 28 is separated from the intramedullary pin at the frangible connection 40, then pulled back into the bone. Alternatively, the removable drill tip 28 does not have to be separated from the pin. In one embodiment, the intramedullary pin 92 has a threaded portion by which it is anchored in anchored in the bone. As shown in FIG. 9C, cerclage wire 95 is used to stabilize the fracture. Intramedullary pin 92 can include a plurality of frangible connections 40.

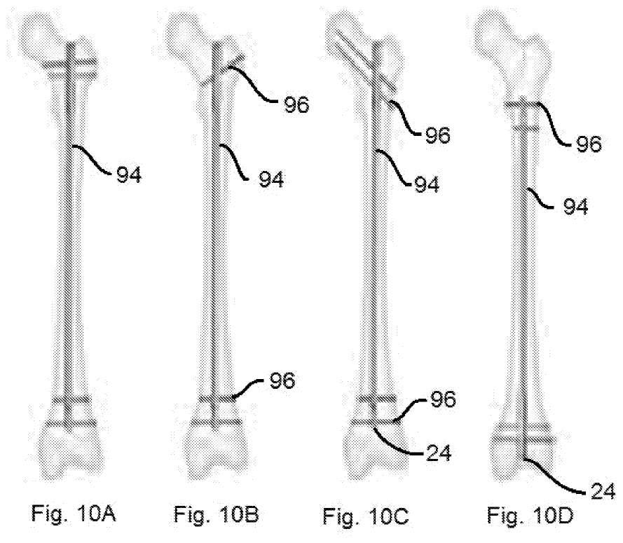

FIGS. 10A-10D are locking intramedullary nails with a removable drill bits. Locking is used to prevent collapse or rotation in inherently unstable fractures. Locking of the nails uses bolts, screws, or the like on each end of the nail to fix the nail to the bony cortex and prevent rotation among the fragments. Locking intramedullary nails 94 are shown in their installed condition. The locking intramedullary nails 94 have had their removable drill tips removed leaving ends 24. It should be noted that is some embodiments, the removable drill tip can be left in place. Locking intramedullary nails 94 have holes at either end. A hole is drilled into the bone at right angle to the nail. The hole passes through the hole in the nail and then drills the opposite cortex. When a bolt 96 is passed through this, it engages cortices on either side while engaging the nail hole too, thus locking nail and bone together. The introduction of paired holes which align at right angles to the long axis of a nail permits cross locking to give axial and rotational stability. Holes may be round, as they are usually at the distal end, to accommodate a screw or bolt 96. The holes are slightly bigger than the thread diameter of either bolt to allow smooth gliding through the nail. Some proximal holes may instead be oval-shaped slots. This permits slight axial movement of the bone but still prevents rotation. Proximal locking is achieved by passing bolts 96 through the aligned holes across the nail guided by a jig which attaches into the top of the nail.

According to one embodiment of the invention, the screws or bolts 96 have a removable drill tip and a separable connection. In this manner, the hole for the bolt 96 can be drilled and the drill tip can be removed in a manner similar to the fixator devices discussed above.

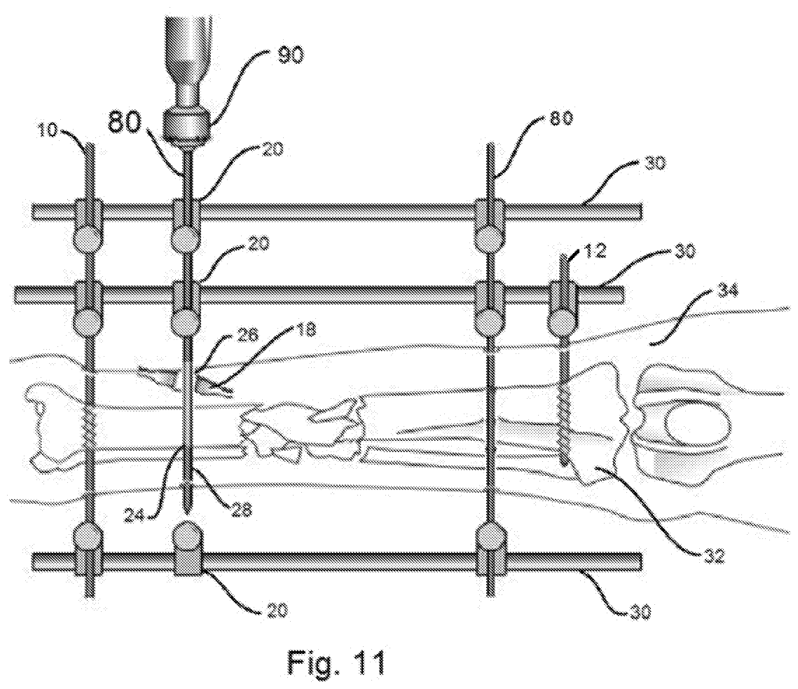

FIG. 11. is a broken bone with a partially installed external fixator pin 80 of an external skeletal fixation system using both threaded and non-threaded fixation pins. A drill 90 is used to install external fixator pin 80. According to one aspect of the invention, a second connecting bar 30 and clamp 20 is used as a drill guide. The external fixator pin 80 is guided into the limb via an incision 18. As shown, the removable drill tip 28 is still attached to the external fixator pin 80 that is being installed by the drill 90. Once the pin 80 passes through the bone 32 via the hole drilled by the removable drill tip 28, the removable drill tip 28 is removed from the external fixator pin 80. Once the removable drill tip 28 is removed from the external fixator pin 80, an end 24 of the external fixator pin 10 remains.

While this invention has been described by reference to a preferred embodiment, it should be understood that numerous changes could be made within the spirit and scope of the inventive concepts described. Multiple variations and modifications are possible in the embodiments of the invention described here. Although certain illustrative embodiments of the invention have been shown and described here, a wide range of modifications, changes, and substitutions is contemplated in the foregoing disclosure. In some instances, some features of the present invention may be employed without a corresponding use of the other features. Accordingly, it is intended that the invention not be limited to the disclosed embodiment, but that it have the full scope permitted by the language of the following claims.

We claim:

1. A fixator pin comprising:
a shaft having an attachment portion configured to be inserted in a bone;
a removable drill tip; and
a connection between the shaft and the removable drill tip.

2. The fixator pin according to claim 1, wherein the connection is one of a frangible connection, a cutable connection, a brittle portion, and a threaded connection.

3. The fixator pin according to claim 2, wherein the connection is proximate to the attachment portion.

4. The fixator pin according to claim 2, wherein the connection is longitudinally spaced from the attachment portion.

5. The fixator pin according to claim 1, wherein the attachment portion is threaded.

6. The fixator pin according to claim 1, wherein the attachment portion is unthreaded.

7. The fixator pin according to claim 1, wherein the removable drill tip comprises a fluted drill portion.

8. The fixator pin according to claim 2, wherein a thread of the threaded is a same direction as a thread of the attachment portion.

9. The fixator pin according to claim 1, further comprising a plurality of connection areas.

10. The fixator pin according to claim 1, wherein the fixator pin is one of an external fixator pin, an intramedullary pin, an intramedullary nail, a Schanz screw, an inter-locking nail, a Küntscher nail, a half pin, a centrally threaded full pin, a Steinmann pin, a smooth transfixation pin, or a positive or negative profile transfixation pin.

11. The fixator pin according to claim 1, wherein the shaft is configured to be attached to a first connecting bar at a first end.

12. The fixator pin according to claim 11, wherein the shaft is configured to be attached to a second connecting bar at a second end opposite the first end.

13. The fixator pin according to claim 1, wherein the shaft is configured to be secured with a bolt.
